(19) United States
(12) Patent Application Publication
(10) Pub. No.: US 2004/0034247 A1
(43) Pub. Date: Feb. 19, 2004

(54) Compositions and methods for inhibiting vinyl aromatic monomer polymerization

(76) Inventor: Sherif Eldin, Houston, TX (US)

Correspondence Address: DUANE MORRIS, LLP, ATTN: WILLIAM H. MURRAY, ONE LIBERTY PLACE, 1650 MARKET STREET, PHILADELPHIA, PA, 19103-7396, US

(21) Appl. No.: 10/222,033

(22) Filed: Aug. 16, 2002

(51) Int. Cl. C07C 50/04; C07C 69/00

(52) U.S. Cl. 560/4; 552/304

(57) ABSTRACT

A synergistic composition and method of use are disclosed. The compositions comprise a quinone methide derivative, a hydroxylamine compound and a catechol compound. These compositions demonstrate synergism at inhibiting the polymerization of a vinyl aromatic monomer. Preferably the quinone methide derivative is 2,6-di-tert-butyl-4-benzylidene-cyclo-2,5-dienone, the hydroxylamine compound is N,N-bis(hydroxypropyl)hydroxylamine, and the catechol is 4-tert-butyl catechol.

### FIELD OF THE INVENTION

The present invention pertains to compositions for use in inhibiting vinyl aromatic monomer polymerization.

### BACKGROUND OF THE INVENTION

Common industrial methods of producing vinyl aromatic monomers typically include separation and purification processes such as distillation to remove unwanted impurities. Unfortunately, purification processes carried out at elevated temperatures result in an increased rate of undesired polymerization. Distillation is generally carried out under vacuum to minimize loss of monomer. The presence of oxygen, which is typically excluded from vinyl aromatic monomer distillation, will also promote polymerization of the monomer. However, in some cases, the presence of oxygen with some inhibitors can produce an effective inhibitor system.

This polymerization results not only in loss of desired monomer end-product, but also in the loss of production efficiency caused by polymer formation, viscosity increase and/or agglomeration of polymer on process equipment. Thermal polymerization, which typically occurs during distillation of vinyl aromatic monomer, results in the formation of normal (i.e., linear) polymer. However, the presence of divinyl benzene may lead to cross-linked polymer.

The compounds generally used commercially to inhibit polymerization of vinyl aromatic monomers are of the dinitrophenol family. For example, U.S. Pat. No. 4,105,506, Watson et al. teaches the use of 2,6-dinitro-p-cresol as a polymerization inhibitor for vinyl aromatic compounds. U.S. Pat. No. 4,466,905, Butler et al. teaches that a combination of 2,6-di-nitro-p-cresol and p-phenylenediamine will inhibit polymerization in a distillation column when oxygen is present. U.S. Pat. No. 4,774,374, Abruscato et al. teaches compositions for inhibiting the polymerization of vinyl aromatic compounds. The composition is an oxygenated product of the reaction of N-aryl-N′alkyl-p-phenylenediamine with oxygen. U.S. Pat. Nos. 5,426,257 and 5,489,718, Arhancet, teach methods and compositions for inhibiting the polymerization of vinyl aromatic monomers comprising an oxime compound and a hydroxylamine compound and/or a phenylenediamine.

The use of hydroxylamine compounds for preventing polymerization of vinyl aromatic compounds is disclosed in U.S. Pat. No. 2,965,685, Campbell. Their use in combination with phenylenediamines is disclosed in U.S. Pat. Nos. 5,396,004 and 5,510,547, Arhancet et al.

U.S. Pat. Nos. 4,003,800 and 4,040,911, Bacha et al., teach methods for inhibiting the polymerization of styrene utilizing a quinone alkide compound without or with a hindered phenol compound, respectively. Preferably, the quinone alkide is a methide such as 2,6-di-t-butyl-4-methenyl quinone methide.

U.S. Pat. No. 5,616,774, Evans et al., discloses processes and compositions for inhibiting the polymerization of vinyl aromatic monomers using a 7-aryl quinone methide. U.S. Pat. No. 5,583,247, Nesvadba et al., teaches inhibiting the polymerization of ethylenically unsaturated monomers with a 7-substituted quinone methide.

There are a number of effective treatments for polymerization control that contain a mixture of inhibitors. These inhibitors tend to react independently, or synergistically with each other, in order to provide inhibition of polymerization. It is usually the exception to find polymerization inhibitors that are antagonistic. Catechols and quinone methides are examples of polymerization inhibitors that are antagonistic. Environmental nitrogen oxides reduction concerns, and the need to replace toxic polymerization retarders such as dinitrophenols with non-toxic materials increases the importance for the use of quinone methides for controlling polymerization. The widespread use of the catechol tert-butyl catechol in inhibiting vinyl monomer polymerization, and the antagonistic effect described above make this difficult. In accordance with the present invention, it has been discovered that hydroxyl amines are capable of reversing the antagonistic effect between catechols and quinone methides.

### DETAILED DESCRIPTION OF THE INVENTION

Compositions comprising a quinone methide derivative, a hydroxylamine and a catechol are provided. These compositions have utility in vinyl aromatic monomers for inhibiting the unwanted polymerization therein.

The quinone methide derivatives generally have the formula:

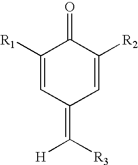

wherein:

R₁ and R₂ are independently H, C₁ to C₁₈ alkyl; C₅ to C₁₂ cycloalkyl; or C₇ to C₁₅ aryl, hydroxy, nitro, amino, carboxy, or mixtures thereof.

Preferably, R₁ and R₂ are methyl, tert-butyl, tert-amyl, tert-octyl, cyclohexyl, α-methylbenzyl or α,α-dimethylbenzyl; with tert-butyl, tert-amyl or tert-octyl most preferred.

R₃ is preferably H, alkyl, aryl, or aryl substituted with C₁ to C₆ alkyl, alkoxy, hydroxy, nitro, amino, carboxy, or mixtures thereof.

Means for preparing these compounds may be found in U.S. Pat. No. 4,032,547, the contents of which are wholly incorporated by reference to herein.

Preferably, the quinone methide derivatives are 4-benzylidene-2,6-di-tert-butyl-cyclohexa-2,5-dienone (QM-1) or 2,6-di-tert-butyl-4-(3,5-di-tert-butyl-4-hydroxy-benzylidene)-cyclohexa-2,5-dienone (QM-2).

The hydroxylamine compounds useful in the present invention generally have the formula:

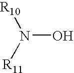

wherein R₁₀ and R₁₁ are the same or different and are hydrogen, alkyl, aryl, alkaryl, aralkyl, or hydroxyalkyl groups and preferably have about three to about twenty carbon atoms. The preferred hydroxylamine compound is N,N-bis(hydroxypropyl) hydroxylamine.

The catechols generally have the formula:

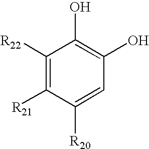

wherein R₂₀, R₂₁, and R₂₂ are the same or different and are e.g., hydrogen, alkyl, alkoxy, hydroxy, nitro, amino, or carboxy groups.

The compositions of the present invention are effective at inhibiting polymerization of vinyl aromatic monomers under processing conditions. These processing conditions include but are not limited to preparation, purification, distillation and vacuum distillation processes.

Styrene, for example, is typically processed at temperatures between 80° and 150° C. The compositions of the present invention are effective at inhibiting the polymerization of styrene over this range of temperatures.

The vinyl aromatic monomers that are treated by the compositions of the present invention include but are not limited to styrene, bromostyrene, divinylbenzene, and α-methylstyrene. The compositions of the present invention are particularly efficacious at inhibiting the polymerization of styrene monomer.

The total amount of quinone methide derivative, hydroxylamine compound and catechol used in the methods of the present invention is that amount which is sufficient to inhibit polymerization of vinyl aromatic monomers. This amount will vary according to the conditions under which the vinyl aromatic monomer is being processed, contaminants in the system and the temperature of the system. At higher processing temperatures and higher monomer contamination, larger amounts of the inhibiting composition are required.

For purposes of the present invention, the term “effective inhibiting amount” is that amount which is effective at inhibiting vinyl aromatic monomer polymerization. Preferably, this amount ranges from about 1 part to about 50,000 parts of quinone methide derivative, hydroxylamine compound and catechol compound, per 1 million parts of monomer. Most preferably, this amount will range from about 1 part total to about 10,000 parts per million parts monomer.

Accordingly, it is possible to produce a more effective vinyl aromatic monomer polymerization inhibiting treatment than is obtained by the use of either compound by itself when measured at comparable treatment levels. This synergism or enhanced activity among components allows for the concentration of each of the components to be lowered, and the total quantity of polymerization inhibitor required, particularly at higher temperatures, may be lowered while achieving a commensurate level of polymerization inhibition.

The compositions of the present invention can be introduced into the vinyl aromatic monomer by any conventional method at any point of the processing system, either as separate and individual ingredients or as a combination of ingredients.

The compositions of the present invention may be added to the vinyl aromatic monomer as either a dispersion or as a solution using a suitable liquid carrier or solvent. Any solvent that is compatible with the individual ingredients of the composition and the vinyl aromatic monomer to be treated may be employed. It is often desirable to dissolve the inhibitors in the monomer to which the inhibitor is being added to avoid introducing additional impurities in the monomer.

In order to more clearly illustrate the invention, the data set forth below ere developed. The following examples are included as being illustrations of the invention and should not be construed as limiting the scope thereof.

### EXAMPLES

In accordance with the present invention, tested were a large number of known free radical polymerization inhibitors, antioxidants, and metal deactivators for their ability to reverse or control the antagonistic behavior between a quinone methide (QM-1) and tert-butyl catechol (TBC) (Table 1). It has been discovered that among materials tested, only hydroxylamines provided this benefit.

TABLE 1
Styrene Static Test Run at 120° C. for 1 hr.

| Treatments¹ (ppm Active) | % Polymer |
|---|---|
| QM-1 (197) | 0.87 |
| QM-1/TBC/DEHA (197/3/15) | 0.63 |
| QM-1/TBC/HPHA (197/3/15) | 1.04 |
| QM-1/TBC (197/3) | 1.47 |
| QM-1/TBC/DNBP (197/3/15) | 1.36 |
| QM-1/TBC/EDTA (197/3/15) | 1.39 |
| QM-1/TBC/2AP (197/3/15) | 1.75 |
| QM-1/TBC/BHT (197/3/15) | 1.71 |
| QM-1/TBC/HBA (197/3/15) | 1.55 |
| QM-1/TBC/DETA (197/3/15) | 1.92 |
| QM-1/TBC/MD (197/3/20) | 1.62 |
| QM-1/TBC/SA (197/3/20) | 1.62 |
| QM-1/TBC/HQ (197/3/50) | 2.30 |

¹As shown in Tables 1-4:
QM-1: 4-benzylidene-2,6-di-tert-butyl-cyclohexa-2,5-dienone
TBC: 4-tert-butyl catechol
DEHA: diethylhydroxylamine
HPHA: N,N-dihydroxypropylhydroxylamine
DNBP: 2,4-dinitro-6-iso-butylphenol
EDTA: ethylenediamine tetraacetic acid
2AP: 2-aminophenol
BHT: 2,6-di-tert-butyl-4-methylphenol
HBA: 2-hydroxy benzyl alcohol
DETA: diethylene triamine
MD: metal deactivator
HQ: hydroquinone
OH-TEMPO: 4-hydroxy-2,2,6,6-tetramethyl-piperidinyloxy
SA: salicylaldoxime

This unexpected, synergistic result among quinone methides/TBC/hydroxylamines is shown by a second polymerization test (Styrene Reflux Polymerization Test) in Table 2. The results demonstrate that the addition of the hydroxylamine HPHA caused the performance of QM-1/HPHA/TBC to mirror that of DNBP/HPHA/TBC.

TABLE 2
Styrene Reflux Test Under argon run at 120° C.

| Time | % | % |
|---|---|---|
| min. | QM-1/HPHA/TBC (197/50/3) | DNBP/HPHA/TBC (197/50/3) |
| 0 | 0.00 | 0.00 |
| 30 | 0.05 | 0.04 |
| 60 | 0.21 | 0.18 |
| 90 | 0.37 | 0.37 |
| 120 | 0.55 | 0.76 |
| 150 | 0.85 | 1.11 |
| 180 | 1.21 | 1.63 |
| 210 | 1.57 | 2.43 |
| 240 | 2.75 | 3.34 |
| 270 | 5.64 | 5.36 |
| 285 | 8.35 | 7.87 |

This is in contrast to experiments run without the presence of a hydroxylamine, as shown in Table 3, where DNBP/TBC performed significantly better than QM-1/TBC.

TABLE 3
Styrene Reflux Test Under argon run at 120° C.

| Time | % | % |
|---|---|---|
| min. | QM-1/TBC (200/15) | DNBP/TBC (200/15) |
| 0 | 0 | 0 |
| 15 | 0.08 | 0.05 |
| 30 | 0.45 | 0.24 |
| 45 | 0.84 | 0.39 |
| 60 | 1.28 | 0.5 |
| 75 | 2.2 | 0.72 |
| 90 | 3.13 | 1 |

The OH-TEMPO material, added to the quinone methide/TBC combination did not control the antagonistic behavior between the quinone methide and TBC, as shown in Table 4 below.

TABLE 4
Styrene Reflux Test Under argon run at 120° C.

| | % | |
|---|---|---|
| Time | QM-1/TBC/OH- | % |
| min. | TEMPO (197/3/50) | QM-1/TBC/HPHA (197/3/50) |
| 0 | 0 | 0 |
| 30 | 0 | 0.05 |
| 60 | 0.18 | 0.21 |
| 90 | 0.98 | 0.37 |
| 120 | 1.94 | 0.55 |
| 150 | 4.29 | 0.85 |
| 180 | 5.88 | 1.21 |
| 210 | | 1.57 |
| 240 | | 2.75 |
| 270 | | 5.64 |

While this invention has been described with respect to particular embodiments thereof, it is apparent that numerous other forms and modifications of this invention will be obvious to those skilled in the art. The appended claims and this invention generally should be construed to cover all such obvious forms and modifications which are within the true spirit and scope of the present invention.

I claim:

1. A method for inhibiting the polymerization of vinyl aromatic monomers comprising adding to said monomer an effective polymerization inhibiting amount of a composition comprising (A) a quinone methide derivative having the formula 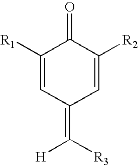
wherein:
R₁ and R₂ are independently C₁ to C₁₈ alkyl; C₅ to C₁₂ cycloalkyl; or C₇ to C₁₅ aryl, hydroxy, nitro, amino, carboxy, or mixtures thereof, and
R₃ is H, alkyl, aryl, or aryl substituted with C₁ to C₆ alkyl, alkoxy, hydroxy, nitro, amino, carboxy, or mixtures thereof;
(B) a hydroxylamine compound having the formula: 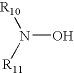
wherein R₁₀ and R₁₁ are the same or different and are hydrogen, alkyl, aryl, alkaryl, aralkyl, or hydroxyalkyl groups and preferably have about three to about twenty carbon atoms;
and (C) a catechol having the formula 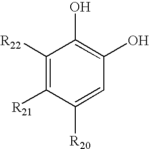
wherein R₂₀, R₂₁, and R₂₂ are the same or different and are hydrogen, alkyl, alkoxy, hydroxy, nitro, amino or carboxy groups.

2. The method as recited in claim 1, wherein said quinone methide derivative is 4-benzylidene-2,6-di-tert-butyl-cyclohexa-2,5-dienone or 2,6-di-tert-butyl4-(3,5-di-tert-butyl-4-hydroxy-benzylidene)-cyclohexa-2,5-dienone.

3. The method as recited in claim 1, wherein said hydroxylamine compound is a hydroxyalkylhydroxylamine.

4. The method as recited in claim 3, wherein said hydroxylamine compound is N,N-bis(hydroxypropyl) hydroxylamine.

5. The method as recited in claim 1 wherein said vinyl aromatic monomer is selected from the group consisting of styrene, bromostyrene, divinylbenzene and α-methylstyrene.

6. The method as recited in claim 1, wherein the temperature of polymerization of said vinyl aromatic monomer ranges from about 80° to about 150° C.

7. The method as recited in claim 1, wherein said composition is added to said monomer in an amount ranging from about 1 to about 50,000 parts per million parts of said monomer.

8. The method as recited in claim 1, wherein said catechol is selected from the group consisting of 4-tert-butyl catechol, 3-methoxycatechol and 3,5-di-tert-butylcatechol.

9. The method as recited in claim 1, wherein (A) is 2,6-di-tert-butyl-4-benzylidine-cyclo-2,5-dienone and (B) is N,N-bis(hydroxypropyl)hydroxylamine.

10. A composition comprising (A) a quinone methide derivative having the formula: 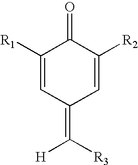
wherein:
R₁ and R₂ are independently C₁ to C₁₈ alkyl; C₅ to C₁₂ cycloalkyl; or C₇ to C₁₅ aryl, hydroxy, nitro, amino, carboxy, or mixtures thereof, and
R₃ is H, alkyl, aryl, or aryl substituted with C₁ to C₆ alkyl, alkoxy, hydroxy, nitro, amino, carboxy, or mixtures thereof,
(B) a hydroxylamine compound having the formula: 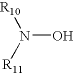
wherein R₁₀ and R₁₁ are the same or different and are hydrogen, alkyl, aryl, alkaryl, aralkyl, or hydroxyalkyl groups and preferably have about three to about twenty carbon atoms;
and (C) a catechol having the formula 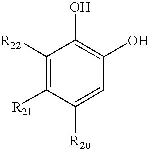
wherein R₂₀, R₂₁, and R₂₂ are the same or different and are hydrogen, alkyl, alkoxy, hydroxy, nitro, amino or carboxy groups.

11. The composition as recited in claim 10, wherein said quinone methide derivative is 4-benzylidene-2,6-di-tert-butyl-cyclohexa-2,5-dienone or 2,6-di-tert-butyl-4-(3,5-di-tert-butyl-4-hydroxy-benzylidene)-cyclohexa-2,5-dienone.

12. The composition as recited in claim 10, wherein said hydroxylamine compound is a hydroxyalkyl hydroxylamine.

13. The composition as recited in 12, wherein said hydroxylamine compound is N,N-bis(hydroxypropyl) hydroxylamine.

14. The composition as recited in claim 10, wherein said catechol is selected from the group consisting of 4-tert-butyl catechol, 3-methoxycatechol and 3,5-di-tert-butylcatechol.

15. The composition as recited in claim 10, wherein (A) is 2,6-di-tert-butyl-4-benzylidene-cyclo-2,5-dienone and (B) is N,N-bis(hydroxypropyl) hydroxylamine.

16. The composition as recited in claim 10, wherein there is synergism among (A):(B) and (C) as a vinyl aromatic monomer polymerization inhibitor.

17. The composition as recited in claim 10, further comprising styrene.

18. The composition as recited in claim 10, wherein (A) is 2,6-di-tert-butyl-4-benzylidene-cyclo-2,5-dienone, (B) is N,N-bis(hydroxypropyl) hydroxylamine and (C) is 4-tert-butyl catechol.
